(19) United States
(12) Patent Application Publication
(10) Pub. No.: US 2007/0292353 A1
(43) Pub. Date: Dec. 20, 2007

(54) Nanoparticles Comprising an Intracellular Targeting Element and Preparation and Use Thereof

(76) Inventors: Laurent Levy, Paris (FR); Abdel Kader Boussaha, Paris (FR); Edouard Andre Panak, Toulouse (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/666,672

(22) PCT Filed: Nov. 4, 2005
(86) PCT No.: PCT/FR05/02758

§ 371 Date: Apr. 30, 2007

(30) Foreign Application Priority Data
Nov. 5, 2004 (FR) 0411806

(51) Int. Cl. A61K 47/48; A61K 41/00; A61K 49/00; A61P 35/00

(52) U.S. Cl. 424/9.34; 424/9.3; 977/911

(57) ABSTRACT

The present invention relates to novel activatable particles which can be used in the health field. More specifically, the invention relates to composite particles comprising an intracellular targeting element, which can generate a response when excited, and to the uses thereof in the health field, particularly in relation to human health. The inventive particles comprise a nucleus comprising at least one inorganic and optionally one or more other organic compound(s) and which can be activated in vivo, in order to label or alter cells, tissues or organs. The invention also relates to methods for producing such particles, as well as pharmaceutical and diagnostic compositions containing same.

The present invention relates to novel activatable particles which can be used in the health field. More specifically, it relates to composite particles comprising an intracellular targeting element, which can generate a response when excited, and to the uses thereof in the health field, particularly in relation to human health. The inventive particles comprise a nucleus comprising at least one inorganic or organic compound which can be activated, in order to label or alter cells, tissues or organs. The invention also relates to methods for producing such particles, as well as pharmaceutical and diagnostic compositions containing same.

Over the past 30 years, major advances have been made in the diagnosis and treatment of human cancers. At the same time, biotechnologies and nanotechnologies have opened new avenues of development and given rise to novel treatments of human pathologies. In practice, chemotherapy is the most widely used method for treating a wide range of cancers. However, chemotherapy has certain limitations and resultant drawbacks. The main drawback of chemotherapy is undoubtedly its toxicity towards the patient's healthy cells, which drastically restricts the doses of drug that can be used to destroy the cancer cells. With the aim of providing a more efficient chemotherapeutic approach, research has focused on specifically targeting chemotherapy drugs to the diseased cells, since cancer cells are recognized as target cells by virtue of the molecules present at their surface (Schally et al., 1999, J. Endocrinol., 141:1; Nagy et al., 1996, Proc. Natl. Acad. Sci. USA, 93:7269; Emons et al., 1993, J. Clin. Endocrinol. Metab., 77:1458).

Since 1950, magnetic particles and probes have been identified as a potential means to treat cancers. Studies have shown that hyperthermia (Grittner et al., 1997, Hybridoma, 16:109; Higler et al., 1997, Invest. Radiol., 32:705) generated by magnetic particles coupled to a high frequency magnetic field could be used as an adjuvant in cancer treatment. It has been found that hyperthermic activity (heat produced by the energy of magnetic relaxation of the magnetic material) efficiently destroys the tumor tissue in the vicinity of the particles or probes.

The development of very small magnetic particles (ferrofluids) with high crystallinity has been the next step in the development of magnetically-induced hyperthermia therapy. Said treatment induces a reduction in tumor volume when the particles are injected directly in the tissue. However, said therapy has not demonstrated any tissue or cell specificity.

An approach based on the use of particles that can be activated by applying a magnetic field, is described in U.S. Pat. No. 6,514,481.

Photodynamic therapy (PDT) is another recently developed treatment method, used to treat superficial cancers such as those of the skin or oesophagus (see for example McCaughan, J. S. Jr., Drugs and Aging. 15: 49-68 (1999) “Photodynamic Therapy. A Review”). This treatment is based on the production of free radicals by photosensitive molecules, during exposure to strong UV or laser light. Indeed, the activated molecules convert the surrounding oxygen to free radicals which are highly reactive species producing irreversible damages in cells. The main cellular organelles attacked are the mitochondria, cell and nuclear membranes, lysosomes, etc. The photosensitive molecules are injected by the intravenous route and generally accumulate at higher concentration in tumor tissue. This makes it possible, after a given time, to have a higher concentration in the tissues to be treated than in healthy tissues. When said molecules are exposed to light (having a suitable wavelength), they produce free radicals from oxygen, which then react with vital components of the cell.

Photodynamic therapy nonetheless has some limitations. For instance, patients may develop light sensitivity, which restricts the number of administrations of said therapy in a given individual. Furthermore, the low wavelengths of the light used to activate the photosensitive molecules preclude passage through a large thickness of tissue, which has the advantage of low toxicity towards other tissues, but restricts the indication to superficial cancers (skin and subcutaneous). Other potential problems inherent to the use of photodynamic therapy are linked to the toxicity of the photosensitive molecules and the need, in some cases, to use oxygen to “load” the tissues to be treated.

Another approach using TiO₂ particles has shown that it was possible to generate free radicals from water and oxygen molecules under UV excitation (Shibata et al., Bioscience Biotechnology and Biochemistry 62:2306-2311 (1998)). This approach has been used in in vitro and in vivo models of bladder cancer.

An approach based on the use of a novel class of particles, designated NanoXRay, activatable by X rays or by UV and which, after activation, can generate free radicals or heat, is described in application FR 04 05036. Said particles can induce a therapeutic or diagnostic response in vivo, even in deep tissues.

The present invention provides improvements to the therapeutic or diagnostic nanoproducts, such as those mentioned hereinabove.

More specifically, within the scope of the present invention, the inventors sought to minimize the potential toxicity of the nanoparticles which can generate a response under activation, such as those described in the aforementioned prior art, by developing novel activatable nanoparticles, i.e., which can label, alter or destroy cells, tissues or organs, even at low concentrations, in vivo, in vitro or ex vivo. These objectives have been attained through the development of novel compounds useful in therapy and/or diagnostics (for example in imaging), particularly in humans, specifically recognizing an intracellular molecule or structure. The inventive particles are applicable to any type of tissue, superficial or deep, in any mammal.

A first aspect of the invention thus relates to biocompatible composite nanoparticles, comprising:
a nucleus comprising at least one inorganic or organic compound activatable by excitation,
optionally, a biocompatible coating, and
at least one targeting molecule, preferably exposed at the particle surface, displaying affinity for an intracellular molecule or structure.

Another object of the invention relates to a method for preparing nanoparticles such as defined hereinabove comprising:
formation of a nucleus comprising one or more compounds such as defined hereinabove,
optional coating of the nucleus,
grafting of at least one targeting molecule displaying affinity for an intracellular molecule or structure at the surface of said particle so formed, optionally coated and, optionally
grafting of at least one surface targeting element enabling specific targeting to biological cells or tissues.

According to another aspect, the invention is based on pharmaceutical or diagnostic compositions, comprising nanoparticles such as defined hereinabove or which can be obtained by the hereinabove method.

Another object of the invention is based on the use of compositions and nanoparticles such as defined hereinabove, in combination with a suitable source of excitation (e.g., light, radiation, an external field, ultrasound, etc.), in order to label, destroy (in a targeted manner), detect or visualize cells, tissues or organs in vitro, ex vivo or in vivo, and on the corresponding methods.

In the spirit of the invention, the term “composite nanoparticle” refers to any synthetic complex of the particle or nanoparticle aggregate type, of small size, generally less than 1000 nm. The shape thereof can vary, for example round, flat, elongated, spherical, oval, and the like. Preferably the shape is essentially spherical. The shape can be determined or controlled by the method of production, and adapted by the person of the art according to the desired applications.

The shape of the particles does not have a major influence on the properties thereof, in particular on the yield of free radicals or heat production or on the nature of the emitted vibrations. However, the shape can influence the “biocompatibility” of the particles. Thus, for pharmacokinetic reasons, nanoparticles having an essentially spherical or round shape are preferred. Also, nanoparticles having a quite homogeneous shape are preferred.

In a preferred manner, the size of the nanoparticles according to the invention is typically comprised between approximately 4 and 1000 nm. The size of the objects must ideally be small enough to enable them to diffuse in the body (tissues, cells, blood vessels, etc.), essentially without being captured by macrophages (phagocytosis) and without causing significant obstruction.

The nanoparticles according to the invention must be biocompatible, that is to say, they must be able to be administered to an organism, typically a mammal. Said biocompatibility can be ensured for example by the nature of the compounds which make up the particle and/or by the optional coating.

Nucleus

As indicated earlier, the inventive particles comprise a nucleus containing at least one type of inorganic or organic compound having particular properties, optionally covered with a coating.

A compound which can enter into the composition of the particle nucleus is an inorganic or organic compound (or a mixture of compounds) which can generate a response under excitation. A compound adapted to the present invention can for example have magnetic properties, in which case the particle undergoes a change in orientation under the influence of a magnetic field. Another adapted compound can absorb X rays, laser light or UV light and emit a response such as UV-visible energy, heat or free radicals. Another type of adapted compound can be sensitive to ultrasounds and emit heat or a specific vibration or can be sensitive to alternating magnetic fields or to microwaves and generate heat, etc. The main function of said inorganic or organic material(s) is to react to a stimulus and to generate a signal in response to said stimulus.

Compounds Sensitive to a Magnetic Field

The compounds sensitive to a magnetic field which can enter into the composition of the nucleus of an inventive particle are typically inorganic compounds. Such compounds are for example non-oxide metals, metal oxides or mixed metal oxide compounds, enabling physical rotation of the particle under the effect of a magnetic field. It can be for example a ferrous or ferric oxide, a cobalt oxide or a mixed iron/cobalt oxide.

Compounds Sensitive to X Rays

The compounds sensitive to X rays which can enter into the composition of the nucleus of an inventive particle are advantageously inorganic compounds. Said compounds are preferably in the form of oxide, hydroxide, sulfoxide or salt, advantageously doped with a doping agent, preferably selected in the group consisting of the rare earth elements (as described in FR 04 05036). For example they can be selected in the group consisting of Y₂O₃, (Y,Gd)₂O₃, CaWO₄, GdO₂S, LaOBr, YTaO₃, BaFCI, Gd₂O₂S, Gd₃Ga₅O₁₂, Rb₃Lu(PO₄)₂ and Cs₃Lu(PO₄)₂. The doping agent used is advantageously a rare earth element selected for example from among Gd, Eu, Tb, Er, Nb, Pr and Ce.

Metallic compounds, in particular non-oxides, can also be used for their X ray absorption and heat emission property. Metallic compounds having such properties are for example Au, Pb or a mixture of amorphous materials and metallic compounds.

Molecules containing atoms which are sensitive to X rays can also be used.

It shall be understood that other inorganic compounds, metals, oxides, hydroxides, sulfoxides or salts and doping agents can be envisioned by the man of the art to form the nucleus of the inventive particles. It shall be understood that several metals, oxides, hydroxides, sulfoxides or salts and/or doping agents can be used as a mixture in the nucleus or nuclei of a same inventive particle.

Compounds Sensitive to UV-Visible IR Light

The compounds sensitive to UV-visible light which can enter into the composition of the nucleus of an inventive particle are advantageously inorganic in nature and can be selected in the group consisting of semiconductor compounds, such as in particular TiO₂, ZnO and, without this being limiting, CdS, CdSe, CdTe, MnTe and mixed solutions (for example CdZnSe, CdMnSe, etc.), optionally doped with a rare earth element (as described in FR 04 05036). The compound(s) sensitive to UV-visible light used can also be organic compounds/molecules which can produce heat or free radicals under the effect of UV light.

Compounds Sensitive to Laser Light

A compound sensitive to laser light which can enter into the composition of the nucleus of the inventive nanoparticles is preferably a compound or a mixture of photosensitive compounds/molecules of an organic or inorganic nature. Such compounds can for example be biological, chemical molecules or a mixture of same. The compound can be a semiconductor compound or a mixed solution, optionally doped with a rare earth element. The activated molecules (under the effect of laser light) convert the surrounding oxygen or other molecules to free radicals or produce heat.

Non-limiting examples of molecules which can be used are haematoporphyrin, mTHPC, chlorine, mono-L-aspartylchlorine, phthalocyanin, etc. Other organic compounds which can be used in the scope of the present invention are, for example, semiconductors (ZnO, TiO₂, etc.), metals (Au, etc).

Compounds Sensitive to Other Types of Radiation

The compounds sensitive to other types of radiation, which can enter into the composition of the nucleus of the inventive nanoparticles, are preferably selected from among a compound or a mixture of compounds of organic or inorganic nature which enable absorption of radiation of the type high frequency, ultrasound, radio waves, etc. or interaction with same.

Such compounds are for example composed of semiconductor, magnetic, insulating materials or a mixture of same.

As indicated earlier the activated compounds can for example generate heat or vibrations.

Generally, the efficacy or the properties of the particles can be adapted by the person of the art by changing the relative amount of the different types of compounds, the overlap between the emission and absorption spectra of the compounds, the crystal structure of the materials, the area of contact between an organic compound and water and/or the distance between the compounds

In the nucleus of the inventive particles, the inorganic or organic compound(s) can be arranged or organized in different ways. For instance, a first compound, preferably inorganic, can form the core of the nucleus, and a second compound (inorganic or organic) can form a mantle or nanoparticles at the surface of the core. Several compounds making up the nucleus can also be arranged in multiple successive layers, a first inorganic compound preferably forming the internal layer (core). The core of the nucleus formed by the first inorganic compound typically has a size comprised between approximately 5 and 50 nm, for example between 7 and 40 nm, and/or the mantle formed by the second compound at the surface of the core has a thickness typically comprised between approximately 1 and 30 nm, for example between 2 and 25 nm.

The compounds of the nucleus can also be present in the form of a mixture of nanoparticles. Said nanoparticles can have various sizes and shapes. In another variant embodiment, the inorganic compounds of the nucleus are present in the form of at least two nuclei in contact with each other.

The person of the art can therefore adapt the properties of the particles by varying the aforementioned parameters, for example according to the planned uses (diagnostic, therapeutic, etc.).

It is understood that, in addition to the different types of compounds described hereinabove, the inventive particles can comprise other molecules, compounds or structure or surface materials, intended to improve their stability, property, function, specificity, etc.

Coating

As indicated earlier, the nanoparticles according to the invention can additionally comprise a coating. Advantageously, such a coating preserves the integrity of the particles in vivo, ensures or improves the biocompatibility thereof, and facilitates the functionalization thereof (for example with spacer molecules, biocompatible polymers, targeting agents, proteins, etc.).

The coating can be composed of any inorganic or organic, amorphous or crystalline structure. In order to preserve the activity of the inventive particles, according to the nature of the nucleus, it may be desirable that the coating allow the diffusion of small molecules and/or free radicals. In particular, it is important that the coating allow the passage of water (or O₂) and the radical form thereof after transformation when the nanoparticle comprises an organic compound which can react with it. This can be accomplished by using materials which are porous and/or a coating layer which has low thickness and is porous. Thus for example, typically a coating is employed which has a porosity comprised between 0.2 and 10 nm. In addition, the coating has a thickness generally comprised between approximately 0.1 and 50 nm, for example between 0.2 and 40 nm.

In general, the coating can be non-biodegradable or biodegradable. Examples of non-biodegradable coatings used are one or more materials selected in the group consisting of silica, agarose, alumina, a saturated carbon polymer or an inorganic polymer, reticulated or not, modified or not (polystyrene for example). Examples of biodegradable coatings are one or more materials selected in the group consisting of biological molecules modified or not, natural or not, a biological molecular polymer modified or not, of natural shape or not, or a biological polymer, such as the saccharide, an oligosaccharide, a polysaccharide, polysulfated or not, for example dextran. The aforementioned materials or compounds can be used alone or in mixtures or assemblies, composite or not, covalent or not, optionally in combination with other compounds. Moreover, it is also possible to use any aforementioned material, naturally or artificially water- or lipid-soluble.

The coating preferably comprises one or more compounds selected in the group consisting of silica (SiO₂), alumina, metals (Au, etc.), polyethylene glycol (PEG) or dextran, optionally in mixture(s).

The coating can also contain different functional groups (or spacer segments), allowing any molecule of interest to bind to the surface of the particle.

Examples of useful functional groups are (CH₂)ₙCOOH, in which n is a whole number from 1 to 10. The targeting molecule and/or the surface element can advantageously be grafted to the coating by means of a functional group (CH₂)ₙCOOH of the coating in which n is an integer from 1 to 10.

For example, the molecules grafted to the surface of the particle can be:
a surface targeting agent enabling specific targeting to biological tissues or cells;
a molecule ensuring or improving biocompatibility; or
a molecule enabling the particle to escape the immune system (and in particular to avoid interactions with macrophages and SRE).

In a particular embodiment, the nanoparticles according to the invention comprise a coating to which an intracellular targeting molecule and optionally a surface targeting component are grafted, preferably by means of a spacer segment.

Intracellular Targeting Element

As indicated earlier, the present application provides novel compounds which can be used in therapy and/or diagnostics, in humans or animals, specifically recognizing an intracellular structure or molecule. The recognition specificity of the inventive nanoparticles enables them to label, alter, or destroy cells, tissues or organs, even at low concentrations, particularly in vivo. The products according to the invention therefore have a lower potential risk of toxicity than the products of the prior art.

One object of the invention relates to a nanoparticle such as defined hereinabove, characterized in that it comprises a targeting molecule displaying affinity for a molecule present in a human or animal cell.

The targeting molecule displaying affinity for an intracellular molecule can be a biological or chemical molecule. One such molecule is selected for example in the group consisting of a peptide, polypeptide, nucleic acid, nucleotide, lipid, metabolite, etc. The targeting molecule is preferably an antibody, receptor ligand, ligand receptor or a fragment or derivative of same. It can also be a hormone, sugar, enzyme, vitamin and the like. Specific examples of targeting molecules which can be used are phalloidin, phosphatidylinositol, rhodamine or HPPH, etc. The chosen intracellular targeting molecule crosses the cell membrane, either spontaneously, or due to its association with the other components of the inventive nanoparticles. It shows preferential affinity for a molecule or structure which is present exclusively or almost exclusively in the cytoplasm or nucleus of the target cells. “Preferential binding affinity” shall be understood to mean a binding affinity substantially higher for the intracellular molecule or structure, than for any surface or extracellular molecule.

The intracellular molecule or structure which is the target in the context of the invention can be a biological or chemical structure, for example a biological structure selected from among a molecule of an intracellular membrane such as the Golgi body, endoplasmic reticulum, intracellular vesicles (endosome, peroxisome, etc.) or of a nuclear membrane, etc., a lysosome, a cytoskeletal molecule, a cytoplasmic molecule, a mitochondria, an enzyme (for example a DNA replication, repair, transciption or translation enzyme, a mitochondrial enzyme), a nuclear receptor, a nucleic acid [for example a preRNA, mRNA, tRNA (in particular their anti-codon fragment), rRNA, DNA], a transcription or translation factor, a cofactor (for example ATP, CoA, NAD, NADPH, etc.), a natural substrate (for example O₂ or other substrates or reaction products), etc. The target intracellular molecule or structure in the spirit of the invention, displaying affinity for the targeting molecule, can also be chemical in nature. For example it can be a synthetic substrate artificially injected in a target cell (O₂ or other substrates or reaction products).

The targeting molecule is grafted to the optional coating or to the nucleus of said nanoparticle, that is to say, to the inorganic or organic compound which constitutes said nanoparticle. The molecule is preferably covalently bound to the surface or adsorbed. Said grafting can be achieved for example via molecular hydrocarbon chains of variable length but also via other types of molecules such as polysaccharides, polypeptides, DNA, etc.

The intracellular targeting element enables the development of nanoparticles which can target an intracellular molecule or structure, preferably a vital component of the cell when said nanoparticles are used in therapy and on the contrary, preferably a non-vital component, when they are used in diagnostics. Examples of preferred vital structure targets are the nucleus, mitochondria, substrates (O₂ for example) or reaction products of a metabolic pathway essential for cell survival, the aim being for example to freeze the reaction equilibria and therefore overall cell functioning. As shown in the examples and as reviewed earlier, lower doses of nanoparticles can be employed to achieve the expected therapeutic result, i.e., destruction of the cell, when the nanoparticle comprises both a surface targeting element and a targeting molecule of an intracellular molecule or structure instead of only a surface targeting element (see FIGS. 3 and 4 which respectively concern nanoparticles activated by laser and by a magnetic field).

Rhodamine is used as targeting molecule in the examples of the application. Said molecule displays affinity for the mitochondria naturally present inside cells. Rhodamine enables targeting of the inventive nanoparticles to intracellular mitochondria, promoting cell destruction when exposed to a source of activation, in so far as mitochondria are a vital component of said cell.

Surface Targeting Element

The nanoparticles according to the invention can comprise, in addition to the targeting molecule displaying affinity for an intracellular structure or molecule, a surface element to specifically target biological tissues or cells. Said surface element can be bound to the particles by any means, preferably covalent, optionally by means of a spacer segment. It can be associated with the nucleus, e.g., with an inorganic compound, or with the optional coating, as described hereinbelow.

The surface targeting element can be any biological or chemical structure displaying an affinity for molecules present in the human or animal body. For instance, it can be a peptide, polypeptide, protein, glycoprotein, lipid, and the like. For example it can be a hormone, vitamin, enzyme and the like, and, in general, any ligand of molecules (for example receptors, markers, antigens, etc.). Ligands of molecules expressed by pathological cells, in particular ligands of tumor antigens, hormone receptors, cytokine receptors or growth factor receptors, for example, can be mentioned by way of illustration Said targeting elements can be selected for example in the group consisting of LHRH, EGF, a folate, anti-B-FN antibody, E-selectin/P-selectin, anti-IL-2Rα antibody, GHRH, trastuzumab, gefitinib, PSMA, tamoxifen/toremifen, imatinib, gemtuzumab, rituximab, alemtuzumab, cetximab, an LDL.

The surface targeting element, when present, enables recognition and preferential accumulation of the inventive particles in the cells, tissues or organs of interest, and thereby confines the action to these tissues. Such targeting is especially useful when the particles are administered by the systemic route, for example for deep tissues.

The surface targeting element enabling specific targeting to biological cells or tissues is grafted to the optional coating or to the inorganic or organic compound which constitutes said nanoparticle.

The combined presence, in the inventive nanoparticles, of a targeting molecule displaying affinity for an intracellular molecule or structure and a surface targeting element allowing specific targeting to biological cells or tissues, improves the specificity of recognition of said nanoparticles for their target. This increased specificity of the nanoparticles allows them to label, alter or destroy cells, tissues or organs, even at low concentrations, particularly in vivo, thereby reducing the potential toxicity inherent to the use of any pharmaceutical or diagnostic composition, as indicated earlier.

LHRH is used as surface targeting element in the examples of the invention. Said molecule displays affinity for LHRH receptors present at the surface of cancer cells, particularly in hormone-dependent tumors. LHRH for example enables targeting of breast, ovary or prostate tumor cells. Double targeting of the inventive nanoparticles, via LHRH and rhodamine, to the mitochondria of cancer cells promotes the destruction of said cells after exposure to a source of excitation. As demonstrated in example 4 in particular, the efficacy of the inventive nanoparticles for destroying target cells is increased when double targeting, that is to say, grafting of a targeting molecule displaying affinity for an intracellular molecule or structure and grafting of a surface targeting element, is used.

Method of Production

The nanoparticles according to the invention can be produced by any method known in the field.

An object of the invention relates to a method for producing nanoparticles such as defined hereinabove, comprising:
formation of a nucleus comprising one or more compounds such as defined hereinabove,
optional coating of the nucleus,
grafting of at least one targeting molecule displaying affinity for an intracellular molecule or structure at the surface of said particle so formed, optionally coated and, optionally
grafting of at least one surface targeting element enabling specific targeting to biological cells or tissues.

The materials which compose the nanoparticles of the invention can be produced by different techniques, known to the man of the art. The method can be adapted by the man of the art according to the nature of the compounds employed, and according to the arrangement thereof in the nanoparticles. Alternative methods for producing materials which can be used for the production of the inventive particles are described for example in Nelson et al., Chem. Mater. 2003, 15, 688-693 “Nanocrystalline Y2O3:Eu Phosphors Prepared by Alkalide Reduction” or else in Liu et al., Journal of Magnetism and Magnetic Materials 270 (2004) 1-6 “Preparation and characterization of amino-silane modified superparamagnetic silica nanospheres” as well as in patents FR 04 05036 and U.S. Pat. No. 6,514,481.

The methods for grafting the targeting elements can be carried out for example by following the protocol described in L. Levy et al., “Nanochemistry: Synthesis and Characterization of Multifunctional NanoBiodrugs for Biological Applications.” (Chem. Mater. 2002, 14(9), 3715-3721).

As indicated earlier, the shape of the particles does not have a major influence on the properties thereof, in particular on the yield of free radicals or heat production or on the nature of the emitted vibrations. However, as the shape can influence the “biocompatibility” of the particles, essentially spherical or round shapes and essentially homogeneous shapes, are preferred.

In a preferred manner, and as indicated earlier, the size of the nanoparticles according to the invention is typically comprised between approximately 4 and 1000 nm, preferably between 300 and 1000 nm, even more preferably between 4 and 250 nm. For in vivo applications in humans or animals, nanoparticles having a size comprised between 4 and 100 nm, more preferably between 4 and 50 nm, are more particularly preferred. The size of the objects must ideally be small enough to enable them to diffuse in the body (tissues, cells, blood vessels, etc.), essentially without being captured by macrophages (phagocytosis) and without causing significant obstruction. Advantageously, such effects can be obtained in humans with particles having a size less than 100 nm, preferably less than 50 nm.

The shape and size of the nanoparticles can be easily calibrated by the person of the art implementing the nanoparticle preparation methods according to the invention.

Compositions

Another object of the invention is based on any composition comprising nanoparticles such as defined hereinabove and/or which can be obtained by the method described hereinabove. While not mandatory, the particles in the inventive compositions advantageously have a quite homogeneous size and shape. Generally, the compositions comprise between 0.3 and 3000 mg of particles per 100 ml. The compositions can be in the form of a solid, liquid (particles in suspension), gel, paste, and the like.

A particular object of the invention relates to a pharmaceutical composition comprising nanoparticles such as defined hereinabove and, optionally, a pharmaceutically acceptable excipient or vehicle.

Another particular object of the invention relates to a diagnostic or imaging composition comprising nanoparticles such as defined hereinabove and, optionally, a physiologically acceptable excipient or vehicle

The excipient or vehicle which is employed can be any classical support for this type of application, such as for example saline, isotonic, sterile, buffered solutions, and the like. The compositions according to the invention can also comprise stabilizers, sweeteners, surfactants, and the like. They can be formulated in ampoules, bottles, as tablets, capsules, by using known techniques of pharmaceutical formulation.

Uses

The compositions, particles and aggregates of the invention can be used in many fields, particularly in human or veterinary medicine.

Depending on the duration of exposure to the source of excitation, the particles can enable the destruction of cells or tissues or, simply, a visualization (imaging, diagnostics).

The man of the art can easily adapt the exposure time of the inventive nanoparticles to the source of excitation according to the nature and intensity of said source, depending on whether the destruction of the cells or the visualization thereof is desired. In the context of a therapeutic use, the nanoparticles according to the invention can be exposed to a source of excitation for a time period usually comprised, for example, between one second and two hours, preferably between 30 minutes and one hour, even more preferably for a time period less than or equal to approximately 30 minutes, for example 5, 10 or 15 minutes. In the context of a diagnostic use, the exposure time of the inventive nanoparticles generally ranges from one second to approximately 30 minutes, for example from one minute to approximately 20 minutes or from one second to approximately 5 minutes, or even from one to approximately 60 seconds. It shall be understood that the greater the surface area exposed to the source of activation, the longer the exposure time and that the exposure time is inversely proportional to the intensity of the source of excitation.

A particular object of the invention is based on the use of compositions, or nanoparticles such as defined hereinabove, in combination with a source of excitation adapted to the nanoparticle nucleus, for preparing a medicament intended to destroy target cells.

Another particular object of the invention is based on a method for inducing or causing the lysis or destruction of target cells, in vitro, ex vivo or in vivo, comprising contacting target cells with one or more nanoparticles such as defined hereinabove, during a period of time sufficient to allow the nanoparticles to penetrate inside the target cells and, exposing the cells to a source of activation adapted to the nanoparticle nucleus, said exposure inducing or causing the lysis or destruction of said target cells.

The target cells can be any pathological cells, that is to say, cells involved in a pathological mechanism, for example proliferative cells, such as tumor cells, stenosing cells (smooth muscle cells), or immune system cells (pathological cell clones).

A preferred application is based on the treatment (for example the destruction or functional alteration) of cancer cells. In this regard, a particular object of the invention is based on the use of compositions or nanoparticles such as defined hereinabove (in combination with a source of activation adapted to the nanoparticle nucleus) for preparing a medicament intended to treat cancer.

Another object of the invention relates to a method of cancer treatment, comprising administering to a patient suffering from a cancer a composition or nanoparticles such as defined hereinabove, in conditions allowing the nanoparticles to penetrate inside the cancer cells, and subsequently treating the patient in the presence of a source of excitation adapted to the nanoparticle nucleus which can be selected from among light, radiation or an external field, more particularly from among X rays and UV light, an external magnetic field, ultrasound, etc., leading to an alteration, disturbance or functional destruction of the patient's cancer cells, thereby treating the cancer.

The invention can be used to treat any type of cancer, in particular solid tumors, metastasized or not, for example selected in the group consisting of cancers of the lung, liver, kidney, bladder, breast, head and neck, brain, ovaries, prostate, skin, intestine, colon, pancreas, eye, etc.

The invention can also be used to treat a cardiovascular pathology such as athersclerosis for example or to treat a neurodegenerative pathology selected for example in the group consisting of Alzheimer's disease, Parkinson's disease, Huntington's chorea, amyotrophic lateral sclerosis, multiple sclerosis. The type of nanoparticle (and its associated therapeutic effect) as well as the intracellular targeting molecule and the surface targeting molecule, optionally present, enabling specific targeting to biological cells or tissues, can thus be chosen according to the type of pathological tissue or cell.

The stimuli can be applied at any time after administration of the particles, on one or more occasions, by using any currently available system such as for example a system of radiotherapy or radiography (scanner for example). The particles can be administered by different routes, preferably by systemic or local injection, or orally. Repeated injections or administrations can be given, where necessary.

Examples of radiation and radiation intensity which can be used to excitate the particles comprising an X ray sensitive compound according to the desired diagnostic or therapeutic use are indicated in FR 04 05036 and reviewed below:

In general and in a non-restrictive manner, the following radiation can be applied in different cases to activate the particles:
Superficial X rays (20 to 50 keV): to activate nanoparticles near the surface (penetration of a few millimeters).
Diagnostic X rays (50 to 150 keV).
X rays (ortho voltage) of 200 to 500 keV which can penetrate to a tissue thickness of 6 cm.
   X rays (mega voltage) of 1000 keV to 25,000 keV. For example the excitation of nanoparticles for the treatment of prostate cancer can be carried out via five focused X rays with an energy of 15,000 keV.

The exposure time to the X rays such as described hereinabove can be easily determined by the man of the art according to the desired therapeutic or diagnostic use and the nature of the nanoparticles.

Magnetic fields of 1.5, 4 or 5 Tesla for example, as well as fields greater than 5 Tesla, can also be applied to the inventive nanoparticles comprising a compound sensitive to a magnetic field. The man of the art can choose the magnetic field to be applied and the exposure time according to the desired therapeutic or diagnostic use. Likewise, the man of the art can easily determine the duration and intensity of exposure to laser, UV or ultrasounds according to the planned uses and the nature of the nanoparticles used.

In the diagnostics field, the inventive particles can be used as contrast agent, for detecting and/or visualizing any type of tissue. They can also be used to freeze reaction equilibria and therefore cell functioning.

Thus, an object of the invention is the use of compositions, or nanoparticles such as defined hereinabove, in combination with an adapted stimulus (source of activation of the particles), for producing a composition intended for the detection or the visualization of cells, tissues or organs

Suitable sources of activation are those indicated earlier. The target cells which can be detected or visualized are for example cancer cells.

The term “in combination” indicates that the sought-after effect is obtained when the cells, tissues or organs of interest, having partially incorporated the nanoparticles of the invention, are activated by the defined source. However, it is not necessary for the particles and stimuli to be administered simultaneously, nor according to the same protocol.

The term “treatment” denotes any improvement in pathological signs, such as in particular a reduction in the size or growth of a tumor or a pathological area of tissue, the suppression or destruction of pathological cells or tissues, a slowing of disease progression, a reduction in the formation of metastases, a regression or a complete remission, etc.

The inventive particles can also be used in vitro or ex vivo. Other aspects and advantages of the invention will become apparent in the following examples, which are given for purposes of illustration and not by way of limitation.

### LEGENDS OF FIGURES

FIG. 1 illustrates the principle of double targeting (Module A: extracellular targeting enabling specific recognition of a cell type, organ, biological tissue of the organism to be treated; and Module B: intracellular targeting enabling specific recognition of an intracellular molecule or structure) with nanoparticles activatable by an external field.

FIG. 2 illustrates the different mechanisms of action of the nanoparticles in therapy or diagnostics.

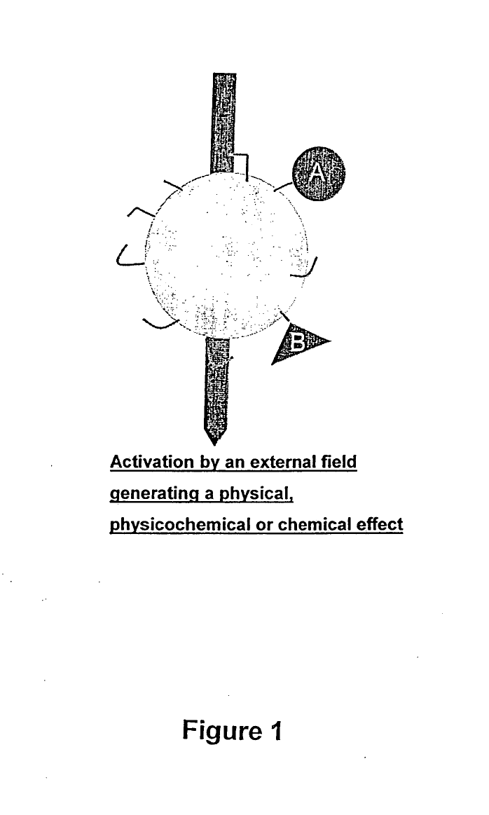

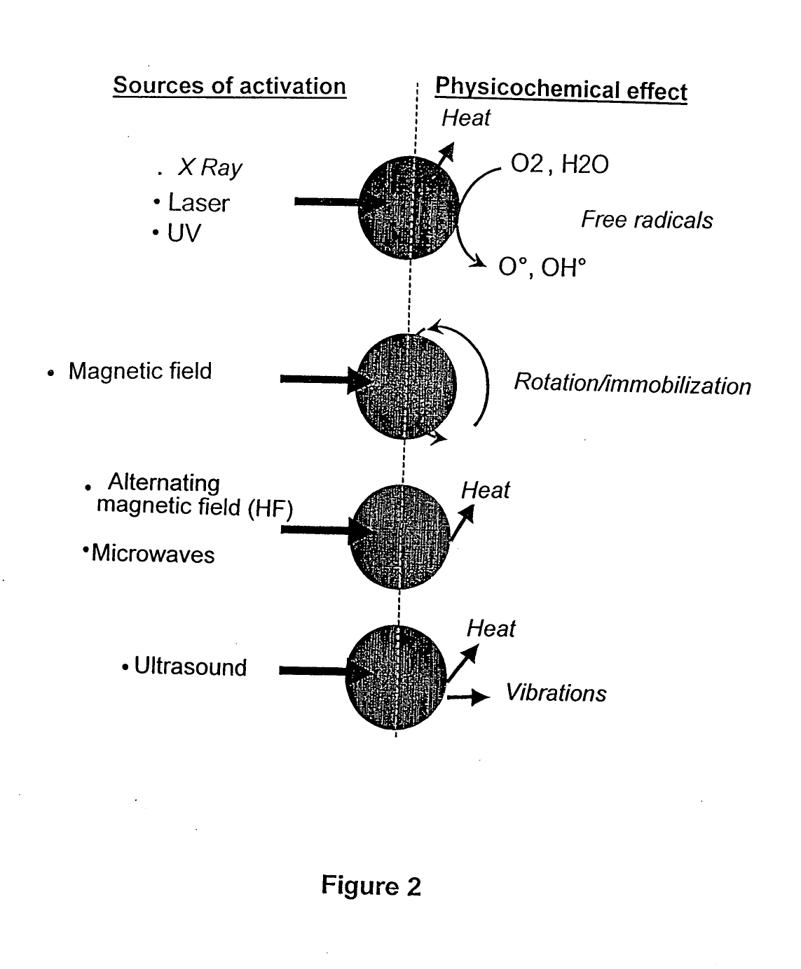

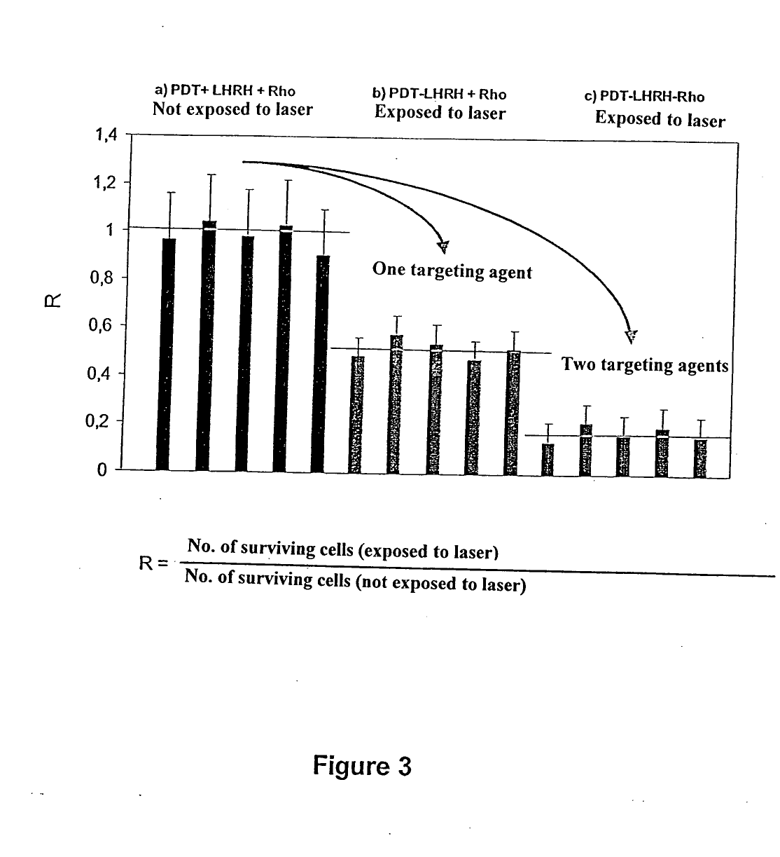

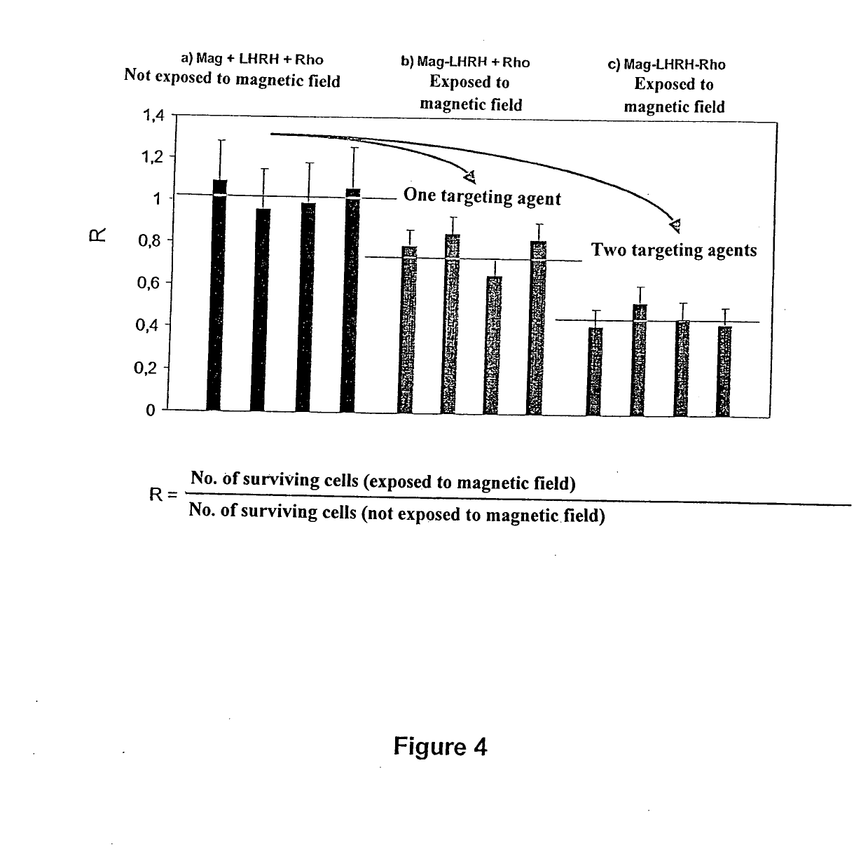

FIG. 3 shows the survival of MCF7 cells (human breast cancer cell line) after incubation with photosensitive nanoparticles of the invention and exposure or not to laser. The experimental conditions were as follows:
a) Nanoparticles were placed in the presence of free rhodamine and free LHRH, dispersed in isotonic solution. The cells were not exposed to laser. The experiment was carried out for 10 minutes in quadruplicate petri dishes.
b) Nanoparticles comprising a targeting molecule with affinity for an intracellular molecule or structure, i.e., rhodamine (targeting molecule displaying affinity for mitochondria), placed in the presence of free LHRH, the nanoparticles and LHRH being dispersed in isotonic solution. The cells were exposed to laser for 10 minutes. The experiment was done in quadruplicate petri dishes.
c) Nanoparticles comprising a targeting molecule with affinity for an intracellular molecule or structure, i.e., rhodamine, and a surface targeting element enabling specific targeting to biological cells or tissues, i.e., LHRH (surface targeting element displaying affinity for cancer cells). The nanoparticles were dispersed in isotonic solution. The cells were exposed to laser for 10 minutes. The experiment was done in quadruplicate petri dishes.

FIG. 4 shows the survival of MCF7 cells after incubation with magnetic nanoparticles of the invention and exposure or not to a magnetic field. The experimental conditions were as follows:
a) Nanoparticles were placed in the presence of free rhodamine and free LHRH, dispersed in isotonic solution. The cells were not exposed to a magnetic field. The experiment was carried out for 10 minutes in quadruplicate petri dishes.
b) Nanoparticles comprising a targeting molecule with affinity for an intracellular molecule or structure, i.e., rhodamine (targeting molecule displaying affinity for mitochondria), placed in the presence of free LHRH, the nanoparticles and LHRH being dispersed in isotonic solution. The cells were exposed to a magnetic field for 10 minutes. The experiment was done in quadruplicate petri dishes.
c) Nanoparticles comprising a targeting molecule with affinity for an intracellular molecule or structure, i.e., rhodamine, and a surface targeting element enabling specific targeting of biological tissues or cells, i.e. LHRH (surface targeting element displaying affinity for cancer cells), the nanoparticles being dispersed in isotonic solution. The cells were exposed to a magnetic field for 10 minutes. The experiment was done in quadruplicate petri dishes.

### EXAMPLES

### Example 1

### Preparation of Photosensitive Nanoparticles Doped with Protoporphyrin IX and Targeted

Photosensitive nanoparticles doped with protoporphyrin IX and targeted were synthesized according to the following protocol:

a) 0.5 g of AOT mixed with 0.5 g of butanol were dissolved in 20 ml distilled water,

b) 30 microliters of DMF and 15 nM protoporphyrin IX were added to the above solution obtained with step a) and mixed,

c) triethoxyvinylsilane (200 microliters) and 3-aminopropyltriethoxysilane (10 microliters) were added to the mixture obtained in b) and stirred for several hours,

d) the solution obtained in c) was dialysed and filtered

e) 3-(triethoxylsilanylpropylcarbamoyl)-butyric acid molecules were added to the nanoparticles of solution d), dispersed in DMF, and the mixture was then stirred for 24 hours,

f) the targeting element displaying affinity for an intracellular molecule or structure (rhodamine) and the surface targeting element (LHRH) were added to the mixture obtained in e) using the method described in L. Levy et al., “Nanochemistry: Synthesis and Characterization of Multifunctional NanoBiodrugs for Biological Applications.” (Chem. Mater. 2002, 14(9), 3715-3721), then

g) the nanoparticles were recovered and their integrity checked.

### Example 2

### Preparation of Three Samples for In Vitro Experiments

Sample a) was composed of nanoparticles placed in the presence of free rhodamine and free LHRH, the nanoparticles, rhodamine and LHRH being dispersed in isotonic solution.

Sample b) was composed of nanoparticles comprising a targeting molecule with affinity for an intracellular molecule or structure (rhodamine), placed in the presence of free LHRH, the nanoparticles and LHRH being dispersed in isotonic solution.

Sample c) was composed of nanoparticles comprising a targeting molecule with affinity for an intracellular molecule or structure (rhodamine) and a surface targeting element enabling specific targeting to biological cells or tissues, (LHRH), the nanoparticles being dispersed in isotonic solution.

The three samples (a, b, c) were added to MCF7 cells (human breast cancer cell line) and incubated for 20 hours at a concentration of 2 pmoles of particles per petri dish. After incubation, cells containing samples a and b were exposed for 10 minutes to a laser source (650 nm). Cell survival was determined 20 minutes after exposure.

The experiment was repeated four times in order to have a statistically significant result. The datas in FIG. 3 show that nanoparticles in sample c) (with double targeting) had greatest efficacy (cell destruction).

### Example 3

### Preparation of Magnetic Nanoparticles

Magnetic nanoparticles were synthesized according to the following protocol

a) 32 g of Fe(NO₃)₃. and 8 g of Fe(Cl)₂ were coprecipitated with sodium hydroxide (13 g) at a temperature of 70° C. with stirring (1 liter reactor);

b) the nanoparticles obtained in a) were rinsed with water (pH 8) and dispersed in an ethanol/water mixture (4:1). TEOS was added in a proportion (mass TEOS=1.2 mass particles) and the mixture was stirred for several hours;

c) 3-(triethoxylsilanylpropylcarbamoyl)-butyric acid molecules were added to the nanoparticles dispersed in DMF and stirred for 24 hours;

d) the targeting element displaying affinity for an intracellular molecule or structure (rhodamine) and the surface targeting element (LHRH) were added using the method described in L. Levy et al., “Nanochemistry: Synthesis and Characterization of Multifunctional NanoBiodrugs for Biological Applications.” (Chem. Mater. 2002, 14(9), 3715-3721).

### Example 4

### Preparation of Three Samples for In Vitro Experiments

Sample a) was composed of nanoparticles placed in the presence of free rhodamine and free LHRH, the nanoparticles, rhodamine and LHRH being dispersed in isotonic solution.

Sample b) was composed of nanoparticles comprising a targeting molecule with affinity for an intracellular molecule or structure (rhodamine), placed in the presence of free LHRH, the nanoparticles and LHRH being dispersed in isotonic solution.

Sample c) was composed of nanoparticles comprising a targeting molecule with affinity for an intracellular molecule or structure (rhodamine) and a surface targeting element enabling specific targeting to biological cells or tissues, (LHRH), the nanoparticles being dispersed in isotonic solution.

The three samples (a, b, c) were added to MCF7 cells and incubated for 20 hours at a concentration of 0.5 picograms of particles per petri dish. After incubation, cells containing samples b) and c) were exposed for 10 minutes to a unidirectional magnetic field (4.7 Tesla). Cell survival was determined 20 minutes after exposure.

The experiment was repeated four times in order to have a statistically significant result. The data in FIG. 4 show that nanoparticles in sample c) (with double targeting) had greatest efficacy (cell destruction).

1-22. (canceled)

23. A biocompatible composite nanoparticle, comprising:
a nucleus comprising at least one inorganic or organic compound activatable by excitation, and
at least one targeting molecule, exposed at the particle surface, displaying affinity for an intracellular molecule or structure.

24. The nanoparticle according to claim 23, wherein the nucleus comprises a metal oxide or a non-oxide metal, enabling physical rotation of the particle under the effect of a magnetic field.

25. The nanoparticle according to claim 23, wherein the nucleus comprises a photosensitive molecule, enabling the production of heat or free radicals under the effect of laser light.

26. The nanoparticle according to claim 23, wherein the nucleus comprises a semiconductor compound or a mixed solution, optionally doped with a rare earth element, or an organic molecule, enabling the production of heat or free radicals under the effect of UV or laser light.

27. The nanoparticle according to claim 23, wherein the nucleus comprises an inorganic compound in the form of an oxide, hydroxide, sulfoxide, salt or mixtures of same optionally doped with a rare earth element, or a non-oxide metal, enabling the production of heat or free radicals under the effect of X rays.

28. The nanoparticle according to claim 23, further comprising a biocompatible coating.

29. The nanoparticle according to claim 28, wherein the biocompatible coating is composed of an inorganic or organic structure, amorphous or crystalline.

30. The nanoparticle according to claim 23, wherein the targeting molecule is a biological or chemical molecule displaying affinity for a molecule present in a human or animal cell such as a peptide, polypeptide, nucleic acid, nucleotide, lipid or metabolite.

31. The nanoparticle according to claim 23, wherein the targeting molecule displays affinity for a molecule of an intracellular or nuclear membrane, a cytoskeletal molecule, a cytoplasmic molecule or a mitochondria.

32. The nanoparticle according to claim 23, wherein the targeting molecule displays affinity for an enzyme, nuclear receptor, transcription or translation factor, cofactor or natural or synthetic substrate artificially injected in a target cell.

33. The nanoparticle according to claim 23, wherein the targeting molecule is an antibody, receptor ligand, ligand receptor or a fragment or derivative of same.

34. The nanoparticle according to claim 23, wherein it comprises a biocompatible coating and the targeting molecule is grafted to the coating or to the nucleus of said particle.

35. The nanoparticle according to claim 23, wherein it additionally comprises a surface element enabling specific targeting to biological cells or tissues.

36. The nanoparticle according to claim 35, wherein the surface element enabling specific targeting to biological cells or tissues is grafted to the nucleus of said particle.

37. The nanoparticle according to claim 35, wherein it comprises a biocompatible coating and the surface element enabling specific targeting to biological cells or tissues is grafted to the coating.

38. The nanoparticle according to claim 23, wherein it comprises a biocompatible coating and the targeting molecule is grafted to the coating via a (CH₂)ₙCOOH functional group in which n is an integer from 1 to 10.

39. The nanoparticle according to claim 35, wherein it comprises a biocompatible coating and the surface element is grafted to the coating via a (CH₂)ₙCOOH functional group in which n is an integer from 1 to 10.

40. The nanoparticle according to claim 23, wherein it has a size comprised between 4 and 1000 nm, preferably between 300 and 1000 nm, even more preferably between 4 and 250 nm, between 4 and 100 nm or between 4 and 50 nm.

41. The nanoparticle according to claim 23, wherein it is essentially spherical in shape.

42. A method for producing nanoparticles comprising (i) a nucleus comprising at least one inorganic or organic compound activatable by excitation, (ii) optionally, a biocompatible coating, and (iii) at least one targeting molecule, exposed at the particle surface, displaying affinity for an intracellular molecule or structure, comprising the steps consisting of:
forming of a nucleus comprising one or more inorganic or organic compounds activatable by excitation,
grafting at least one targeting molecule displaying affinity for an intracellular molecule or structure at the surface of said particle so formed optionally coated.

43. The method of claim 42, wherein the method comprises an additional step of grafting at least one surface targeting element enabling specific targeting to biological cells or tissues.

44. A pharmaceutical composition comprising nanoparticles comprising (i) a nucleus comprising at least one inorganic or organic compound activatable by excitation, (ii) optionally, a biocompatible coating, and (iii) at least one targeting molecule, exposed at the particle surface, displaying affinity for an intracellular molecule or structure.

45. The pharmaceutical composition of claim 44, wherein said nanoparticles have a biocompatible coating.

46. A method for inducing or causing functional alteration, lysis or destruction of target cells, in vitro, ex vivo or in vivo, comprising contacting target cells with one or more nanoparticles comprising (i) a nucleus comprising at least one inorganic or organic compound activatable by excitation, (ii) optionally, a biocompatible coating, and (iii) at least one targeting molecule, exposed at the particle surface, displaying affinity for an intracellular molecule or structure, during a period of time sufficient to allow the nanoparticles to penetrate inside the target cells and, exposing the cells to a source of activation adapted to the nanoparticle nucleus, said exposure inducing or causing the lysis or destruction of said target cells.

47. The method according to claim 46, wherein the target cells are selected from the group consisting of proliferative cells, stenosing cells or immune system cells.

48. The method according to claim 46, wherein the target cells are tumour cells.

49. The method according to claim 46, wherein the source of excitation is a light, a radiation or an external field.

50. A method for treating cancer, comprising administering to a patient suffering from a cancer, one or more nanoparticles comprising (i) a nucleus comprising at least one inorganic or organic compound activatable by excitation, (ii) optionally, a biocompatible coating, and (iii) at least one targeting molecule, exposed at the particle surface, displaying affinity for an intracellular molecule or structure, in conditions allowing the nanoparticles to penetrate inside the cancer cells, and subsequently treating the patient, on one or more occasions, in the presence of a source of excitation adapted to the nanoparticle nucleus leading to an alteration, disturbance or functional destruction of the patient's cancer cells, thereby treating the cancer.

51. The method according to claim 50, wherein the source of excitation is a light, a radiation or an external field.

52. The method according to claim 50, wherein the cancer is selected in the group consisting of lung, liver, kidney, bladder, breast, head and neck, brain, ovaries, prostate, skin, intestine, colon, pancreas, and eye cancer.

53. A method for detecting or visualizing cells, tissues or organs, comprising contacting target cells with one or more nanoparticles comprising (i) a nucleus comprising at least one inorganic or organic compound activatable by excitation, (ii) optionally, a biocompatible coating, and (iii) at least one targeting molecule, exposed at the particle surface, displaying affinity for an intracellular molecule or structure, during a period of time sufficient to allow the nanoparticles to penetrate inside the target cells and, exposing the cells, on one or more occasions, to a source of activation adapted to the nanoparticle nucleus, said exposure inducing or causing the lysis or destruction of said target cells.
